(19) United States
(12) Patent Application Publication
(10) Pub. No.: US 2007/0227914 A1
(43) Pub. Date: Oct. 4, 2007

(54) Suture package

(76) Inventors: Robert J. Cerwin, Pipersville, PA (US); Clifford A. Dey, Rieglesville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/393,503

(22) Filed: Mar. 30, 2006

(51) Int. Cl. A61B 17/06

(52) U.S. Cl. 206/63.3

(57) ABSTRACT

A suture package having a cover member and base member configured so that, when coupled together, they form a continuous channel therebetween. A dispensing port opening through the cover member is aligned with and provides access into the continuous channel. The package also includes a plurality of sutures extending substantially along the length of and within the channel, with an end region of each suture extending out of the channel through the dispensing port opening to a distal end that is removably secured to the package by the holding device. A pathway followed by each suture from the point at which the suture extends out from the suture channel through the dispensing port opening to the point at which the suture is removably secured to the package by the holding device, changes direction by at least 90 degrees.

### BACKGROUND OF THE INVENTION

1. Field of the Invention

The present invention relates to packaging, and more particularly, to packages used for surgical sutures.

2. Background Discussion

Packaging for surgical sutures and needles is well known in the art. Conventional packages serve several useful functions, including protecting the needles and sutures during handling, shipping, and storage, and also to facilitate dispensing of the sutures during surgery or other medical procedures. Known packages may be used for surgical sutures armed with surgical needles, or for unarmed surgical sutures without needles.

One known type of suture package is a tray package having a winding channel. These packages are typically oval in shape with inner and outer walls forming an oval winding channel. Such packages are typically molded from plastic, and are mounted onto a winding fixture with the sutures subsequently wound into the winding channel. One prior art suture package of this type is illustrated in FIGS. 1 and 2, and is described in detail in U.S. Pat. No. 6,047,815, which is incorporated herein by reference in its entirety. Referring to FIG. 1, the package 100 includes multiple suture strands 102 wound and positioned within winding channel 104. As better shown in FIG. 2, the package has a top portion 106 and a bottom portion 108 having an outer wall 110 and inner wall 112 that, when joined together, form the winding channel 104. The sutures exit the winding channel through a dispensing port 113 that is an opening in the top portion 106. The sutures exit the dispensing port and extend to their respective ends 114 where they are held in place by a holding means such as a clip 116 or the like. In these known packages, sutures are dispensed (by pulling on the free ends 114) at an angle A of less than 90 degrees from the path of the suture upon exiting the dispensing port. In other words, the dispensing direction of the sutures (“dispensing direction” as indicated by arrow B) is at a substantially similar angle (as illustrated substantially less than 90 degrees) to the direction at which the sutures exit the dispensing port (“dispensing port direction” as illustrated by arrow C).

With multi-strand suture packages of the type described above, it is important that one be able to readily remove individual suture strands one at a time. With the above-described suture package, however, a typical problem is that pulling on one suture strand causes one or more other strands to follow. In other words, when one suture strand is pulled, others tend to be dragged, or at least partially dragged out with the one suture.

Accordingly, there is a need in the art for a new and improved suture package that better allows a single suture strand to be removed without affecting remaining suture strands in the package.

### SUMMARY OF THE INVENTION

The present invention provides a suture package including a cover member having a top side and a bottom side, a holding device on the top side thereof, and a dispensing port opening therethrough; and a base member having a top side, a bottom side, an outer peripheral wall extending upwardly from the top side about a first peripheral location, and an inner peripheral wall extending upwardly from the top side about a second peripheral location that is inward of the first peripheral location. The base member is coupled with the cover member such that a portion of the bottom surface of the cover member, in conjunction with a portion of the top surface and inner and outer peripheral walls of the base member form a continuous channel therebetween, and such that the dispensing port opening of the cover member is aligned with and provides access into the continuous channel. The package further includes a plurality of sutures extending substantially along the length of and within the channel. An end region of each suture extends out of the channel through the dispensing port opening to a distal end that is removably secured to the package by the holding device. A pathway followed by each suture from the point at which the suture extends out from the suture channel through the dispensing port opening to the point at which the suture is removably secured to the package by the holding device, changes direction by at least 90 degrees.

The channel may be substantially oval or circular in shape, and the inner and/or outer peripheral walls may further consist of a plurality of consecutive wall members having spaces therebetween.

In one embodiment, the pathway changes direction by at least approximately 180 degrees.

In yet another embodiment the holding device of the cover member is positioned inward of the inner peripheral wall. The suture package may further include first and second ends, and first and second side edges, wherein the suture distal ends are held by the holding device such that they can be withdrawn by a user in a direction substantially parallel to the first and second side edges.

In yet another embodiment, the dispensing port is positioned substantially along the first lateral edge of the suture package and the sutures exit the dispensing port in a direction toward the second end of the suture package. The holding device may further be positioned closer to the first end of the suture package than the dispensing port.

Also provided is a suture package having a cover member and a base member positioned adjacent one another and configured so as to form a continuous channel therebetween, and a holding means positioned on a top surface of the cover member. The suture channel has a substantially oval configuration, and the suture cover member has a dispensing port opening therethrough providing access into the channel. The package includes a plurality of sutures extending substantially along the length of and within the channel, with an end region of each suture extending out of the channel through the dispensing port opening to a distal end that is removably secured to the package by the holding device. Each suture exits through the dispensing port and changes direction by at least 90 degrees before being held in place by the holding device.

In one embodiment, the sutures change direction by at least approximately 180 degrees before being held in place by the holding device, and in yet another embodiment the holding device is positioned inward of the channel.

In an alternate embodiment, the suture package further comprises first and second ends, and first and second side edges, wherein the suture distal ends are held by the holding device such that they can be withdrawn by a user in a direction substantially parallel to the first and second side edges. The dispensing port may further be positioned substantially along the first lateral edge of the suture package and the sutures exit the dispensing port in a direction toward the second end of the suture package. In yet another embodiment, the holding device is positioned closer to the first end of the suture package than the dispensing port. The holding device of the package may further be a cantilever arm.

### BRIEF DESCRIPTION OF THE DRAWINGS

FIG. 1 is perspective view of a prior art suture package having surgical sutures therein;

FIG. 2 is an exploded perspective view of the prior art suture package of FIG. 1;

FIG. 3 is a perspective view of a suture package according to the present invention having surgical sutures therein; and

FIG. 4 is an exploded perspective view of the suture package of FIG. 3.

### DETAILED DESCRIPTION OF THE INVENTION

Before explaining the present invention in detail, it should be noted that the invention is not limited in its application or use to the details of construction and arrangement of parts illustrated in the accompanying drawings and description. The invention as illustrated may be implemented or incorporated in other embodiments, variations and modifications, and may be practiced or carried out in various ways.

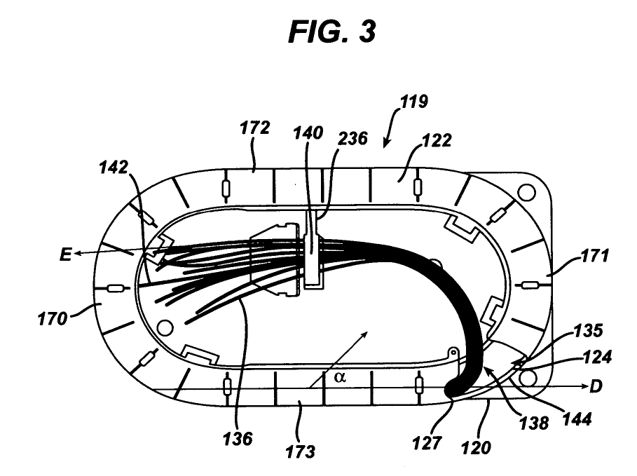

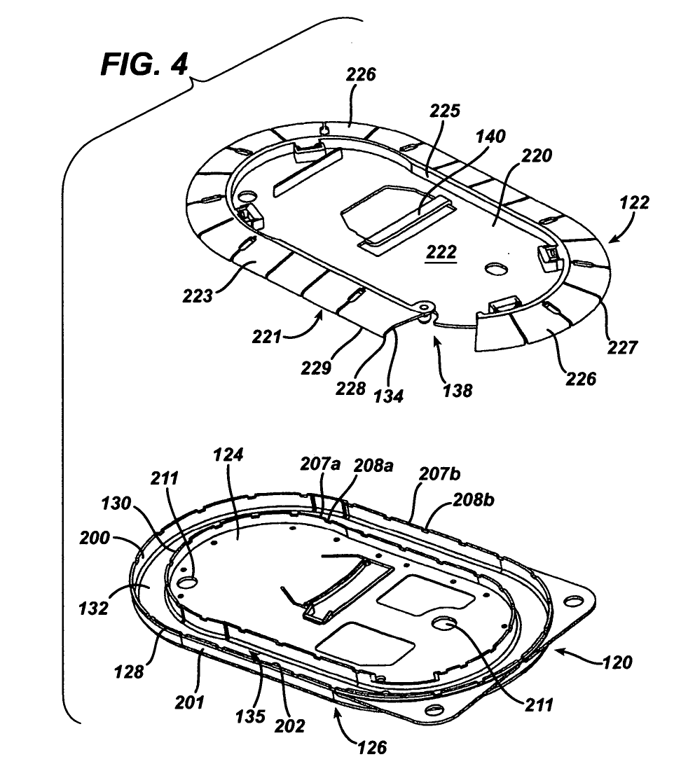

Referring now to FIGS. 3 and 4, one embodiment of a suture package 119 according to the present invention has a first end 170, a second end 171, and first and second side edges 172, 173, and is preferably substantially oval in overall configuration, although other configurations (circular, polygonal, rectangular with rounded corners etc.) may be suitable as well. The suture package further includes a base member or portion 120 and a cover member 122. The base member 120 has a top side 124 and a bottom side 126, an outer periphery 127. Projecting upwardly from the top surface of the base member is an outer peripheral wall 128 and an inner peripheral wall 130. The outer peripheral wall and inner peripheral wall, in conjunction with the winding channel lower surface 132 of the base member and winding channel upper surface (defined by a portion 134 of the lower surface or bottom side 221 of channel cover member 122) define an enclosure or winding channel 135 within which the suture strands 136 are placed. In the illustrated embodiment, the winding channel is substantially oval in shape. The winding channel upper surface of the channel cover member has an opening therein 138, through which the wound suture strands 136 exit the winding channel as shown in FIG. 3.

The upwardly extending outer peripheral member 128 has an inner side 200, outer side 201 and top side 202. The inner and outer peripheral walls 130, 128 may consist of a plurality of members 207a, 207b that extend upwardly from the base member 120, and that may be separated or partially separated by openings or partial openings 208a, 208b. Extending through the base member 120 interior of the interior peripheral wall 207 are one or more winding pin locating holes 211 for locating the base member on the winding head.

The cover member 122 also has a top side 220, a bottom side 221, and includes a first portion 222 that is centrally located and a suture channel cover portion 223 that extends about the peripheral area of the cover member 122. The inner edge 225 of the suture channel cover portion 223 preferably extends upwardly from the top surface of the first portion 222 as illustrated. The suture channel cover portion may further be comprised of multiple cantilevered cover members 226 separated from each other by a space 227. Further, a rim 228 may project downwardly from the outermost peripheral edge 229 of the cover members 226.

The suture channel cover portion 223 is discontinuous around the periphery of the cover member in that there is an opening 138 forming a dispensing port through the suture channel cover portion. Mounted within the first portion 222 of the cover member 122 is a holding device such as a cantilevered clip arm 140.

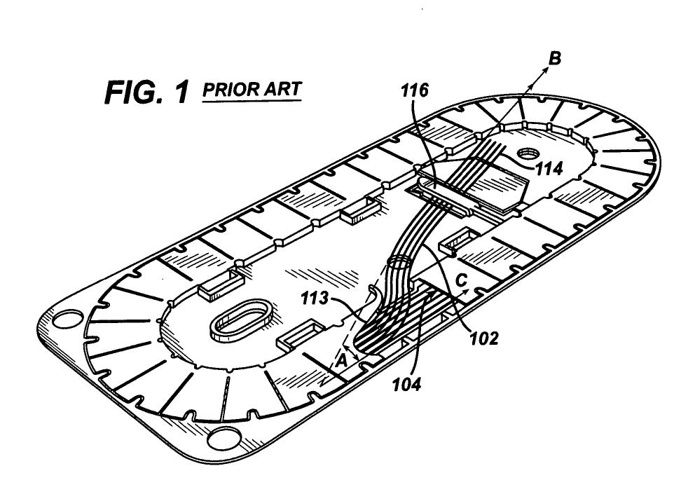

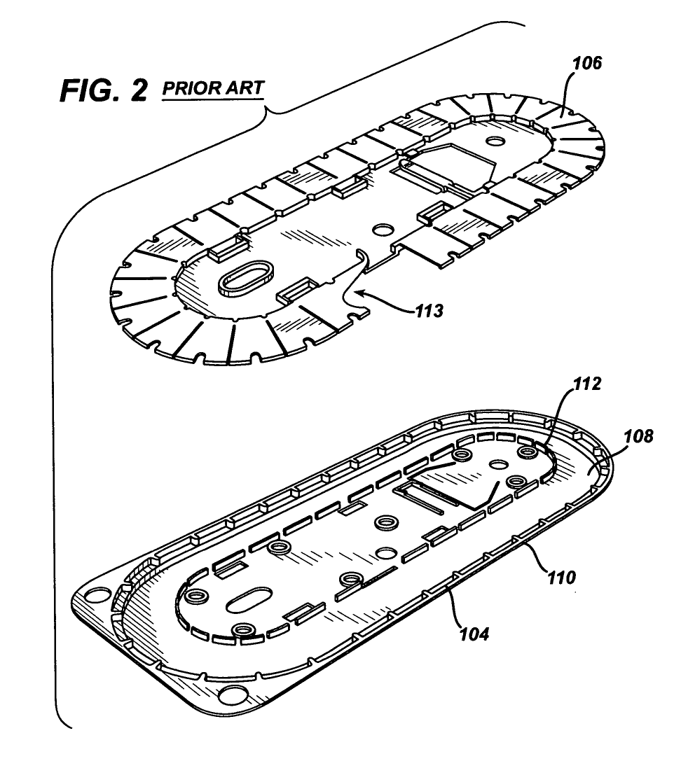

In contrast to prior art suture packages as shown in FIGS. 1 and 2, the cantilevered clip arm 140 of the package shown in FIGS. 3 and 4 is located in a position such that suture strands in the suture package that exit the suture channel through opening 138 must extend in an arc approximating a 180 degree turn before their ends are held in place by clip arm 140, as is shown in FIG. 3. In other words, the direction at which the suture strands 136 exit the opening or dispensing port 138 is shown generally by arrow D, whereas the direction at which they exit the holding element or clip arm 235 is shown generally by arrow E. As is readily apparent in the illustrated embodiment, the suture will travel around an angle α of approximately 180 degrees between the point and direction at which they exit the dispensing port and the point and direction at which they can be removed by the user. Although approximately 180 degrees is the preferred arc, arcs of at least approximately 90 degrees may be suitable as well.

By forcing the suture strand to travel along a pathway representing at least a 90 degree, and preferably approximately 180 degree, bend before being removed entirely from the package, the “dragging” problem associated with prior art packages has been significantly reduced.

It will be apparent from the foregoing that, while particular forms of the invention have been illustrated and described, various modifications can be made without departing from the spirit and scope of the invention. Accordingly, it is not intended that the invention be limited, except as by the appended claims.

What is claimed is:

1. A suture package comprising:
a cover member having a top side and a bottom side, a holding device on the top side thereof, and a dispensing port opening therethrough;
a base member having a top side, a bottom side, an outer peripheral wall extending upwardly from the top side about a first peripheral location, and an inner peripheral wall extending upwardly from the top side about a second peripheral location that is inward of the first peripheral location, the base member being coupled with the cover member such that a portion of the bottom surface of the cover member, in conjunction with a portion of the top surface and inner and outer peripheral walls of the base member form a continuous channel therebetween, and such that the dispensing port opening of the cover member is aligned with and provides access into the continuous channel; and
a plurality of sutures extending substantially along the length of and within the channel, with an end region of each suture extending out of the channel through the dispensing port opening to a distal end that is removably secured to the package by the holding device,
wherein a pathway followed by each suture from the point at which the suture extends out from the suture channel through the dispensing port opening to the point at which the suture is removably secured to the package by the holding device, changes direction by at least 90 degrees.

2. The package according to claim 1, wherein the channel is substantially oval or circular in shape.

3. The package according to claim 2, wherein the inner and/or outer peripheral walls are comprised of a plurality of consecutive wall members having spaces therebetween.

4. The package according to claim 1, wherein the pathway changes direction by at least approximately 180 degrees.

5. The package according to claim 1, wherein the holding device of the cover member is positioned inward of the inner peripheral wall.

6. The package according to claim 5, wherein the suture package further comprises first and second ends, and first and second side edges, and wherein the suture distal ends are held by the holding device such that they can be withdrawn by a user in a direction substantially parallel to the first and second side edges.

7. The package according to claim 6, wherein the dispensing port is positioned substantially along the first lateral edge of the suture package and the sutures exit the dispensing port in a direction toward the second end of the suture package.

8. The package according to claim 7, wherein the holding device is positioned closer to the first end of the suture package than the dispensing port.

9. A suture package comprising:
a cover member and a base member positioned adjacent one another and configured so as to form a continuous channel therebetween, and a holding means positioned on a top surface of the cover member, the suture channel having a substantially oval configuration, and the suture cover member have a dispensing port opening therethrough providing access into the channel;
a plurality of sutures extending substantially along the length of and within the channel, with an end region of each suture extending out of the channel through the dispensing port opening to a distal end that is removably secured to the package by the holding device,
wherein each suture exits through the dispensing port and changes direction by at least 90 degrees before being held in place by the holding device.

10. The package according to claim 1, wherein the sutures change direction by at least approximately 180 degrees before being held in place by the holding device.

11. The package according to claim 9, wherein the holding device is positioned inward of the channel.

12. The package according to claim 11, wherein the suture package further comprises first and second ends, and first and second side edges, and wherein the suture distal ends are held by the holding device such that they can be withdrawn by a user in a direction substantially parallel to the first and second side edges.

13. The package according to claim 12, wherein the dispensing port is positioned substantially along the first lateral edge of the suture package and the sutures exit the dispensing port in a direction toward the second end of the suture package.

14. The package according to claim 13, wherein the holding device is positioned closer to the first end of the suture package than the dispensing port.

15. The package according to claim 9, wherein the holding device is a cantilever arm.
